(19) United States
(12) Patent Application Publication
(10) Pub. No.: US 2025/0325563 A1
(43) Pub. Date: Oct. 23, 2025

(54) METHODS FOR TREATING METABOLIC SYNDROME

(71) Applicants: ADGE PHARMACEUTICALS INC., Mountain View, CA (US); UNIVERSITY OF TARTU, Tartu (EE)

(72) Inventors: Kalev KASK, Mountain View, CA (US); Aleksandr ZARKOVSKI, Tartu (EE); Monika JURGENSON, Kambja (EE)

(21) Appl. No.: 19/258,700

(22) Filed: Jul. 2, 2025

Related U.S. Application Data
(63) Continuation of application No. PCT/US2023/085991, filed on Dec. 27, 2023.
(60) Provisional application No. 63/478,868, filed on Jan. 6, 2023.

(51) Int. Cl. A61K 31/593; A61K 38/26; A61P 3/04; A61P 3/06; A61P 3/10

(52) U.S. Cl. CPC A61K 31/593; A61K 38/26; A61P 3/04; A61P 3/06; A61P 3/10

(57) ABSTRACT

Disclosed herein are methods for treating metabolic syndrome in a subject using a vitamin D receptor agonist. A preferred vitamin D receptor agonist for the present invention is elocalcitol.

### CROSS-REFERENCE TO RELATED APPLICATION(S)

This application is a continuation of PCT/US2023/085991, filed Dec. 27, 2023; which claims the benefit of U.S. Provisional Application Nos. 63/478,868, filed Jan. 6, 2023. The contents of the above-identified applications are incorporated herein by reference in their entirety.

### FIELD OF THE INVENTION

The invention disclosed herein relates generally to methods for treating metabolic syndrome in a subject using a vitamin D receptor agonist.

### BACKGROUND OF THE INVENTION

Metabolic syndrome is a cluster of conditions that occur together, increasing the risk of heart disease, stroke, and type 2 diabetes. The conditions include excess body fat (visceral fat) around the waist, abnormal cholesterol or triglyceride levels (dyslipidemia), increased blood pressure (hypertension), insulin resistance or glucose intolerance, high blood sugar (hyperglycemia), obesity, atherosclerosis, diabetes mellitus, and hepatic steatosis. The activation of NF-κB signaling has been found to be central in the development of metabolic syndrome; thus, NF-κB inhibition offers a potential therapeutic pathway to address the metabolic syndrome. Vitamin D acts as an agonist of a nuclear receptor referred to as Vitamin D receptor that controls gene expression associated with regulation of calcium homeostasis and other cellular functions such as inhibition of upregulated NF-κB signaling. In particular, elocalcitol is a vitamin D analogue and a high-affinity vitamin D receptor agonist that inhibits NF-κB and is thus a good candidate for treating metabolic syndrome.

The prevalence of metabolic syndrome is estimated to be at least 34% in the US and at least 25% worldwide. The total cost of healthcare of cardiovascular and musculoskeletal complications, cancer and neurodegenerative diseases stemming from the metabolic syndrome combined with the lost economic activity is estimated to be in trillions of dollars (Van Saklayen, 2018).

The precise underlying causes of metabolic syndrome vary with both genetic and lifestyle factors contributing significantly. Metabolic syndrome develops when a chronic excessive energy intake triggers imbalances in energy metabolism that gives rise to chronic inflammation that leads to further aggravation of underlying conditions and to emergence of pathological conditions (Chawla et al., 2011).

Since the activation of NF-κB signaling is central to the development of metabolic syndrome by driving the expression of pro-inflammatory cytokines such as TNFα, IL-1β, Il-18, IL6, priming of the NRLP3 inflammasome and the emergence of insulin resistance (Shi et al., 2006; Hotamisligil 2006; He et al., 2016; Hotamisligil, 2017), NF-κB inhibition offers a potential therapeutic pathway to address the metabolic syndrome (Baker et al., 2011).

Vitamin D is a steroid made in the body or obtained from foods that are converted to active agents that regulate absorption of calcium in the gut and functions in proper homeostasis of calcium and phosphate in serum that underlie mineralization of bone for bone growth and remodeling. Vitamin D has been found to play a role in the development and function of multiple organs including immune and nervous systems. Vitamin D acts as an agonist of a nuclear receptor referred to as Vitamin D receptor that controls gene expression associated with regulation of calcium homeostasis and other cellular functions such as inhibition of upregulated NF-κB signaling. Although the natural ligand of the Vitamin D receptor is 1,25 (OH)₂D₃. its actions as a Vitamin D receptor can be mimicked by a large number of synthetic Vitamin D analogues. As with other receptors, agonists need to mimic the action of the natural ligand on its cognate receptor but not necessarily be a structural analogue, i.e., of the same chemical class as the natural agonist. The functional class of Vitamin D agonist includes Vitamin D analogues, but also, by way of example and not exclusion, chemical compounds that are not classified as steroids, peptides, and nucleic acids.

Metabolic syndrome is increasingly common, with up to a third of U.S. adults having metabolic syndrome. There is thus a need for an effective treatment for metabolic syndrome.

### BRIEF DESCRIPTION OF THE DRAWINGS

FIG. 1 shows the mean body weights (±S.D.) of experimental animals in elocalcitol, vitamin D₃ and vehicle groups.

FIG. 2 Shows the average relative body weight loss or gain of experimental animals in elocalcitol, vitamin D₃ and vehicle groups.

FIGS. 3A-3B show average body weight and weight gain data of animals: comparison of average body weights in four groups of mice (LFD, HFD, HFD+vitD and HFD+Eloc) after the 16-week low- or high-fat diet and concomitant 1,25 (OH)₂D₃ or elocalcitol administration to HFD-fed mice, with data expressed as mean+SEM (**p<0.01; ****p<0.001-compared to LFD group; #p<0.05, ###p<0.001-compared to HFD group; one-way ANOVA, n=15 per group) (FIG. 3A); and relative average weight gain dynamics of animals in different experimental groups, with data expressed as mean+SEM (*p<0.05; **p<0.01-HFD+vitD group compared to HFD group; ##p<0.01-HFD+Eloc group compared to HFD group; two-way RM ANOVA, followed by Tukey's multiple comparisons post-tests, n=15 per group) (FIG. 3B).

FIGS. 4A-4C show fat distribution and visceral fat volume changes. FIG. 4D shows the lean mass as calculated from the volume of back muscle. Visceral and subcutaneous fat distribution presented in axial slices of MRI at the level of the kidneys is shown in FIG. 4A; visceral fat volume changes after 16 week of LFD or HFD diets and 1,25 (OH)₂D₃ or elocalcitol treatments is shown in FIG. 4B; and subcutaneous fat volume changes after 16 week of LFD or HFD diets and 1,25 (OH)₂D₃ or elocalcitol treatments is shown in FIG. 4C. The values in FIG. 4B and FIG. 4C are expressed as mean+SEM (*p<0.05; **p<0.01; ***p<0.001; ****p<0.0001 two-way ANOVA, followed by Tukey's multiple comparisons post-test; n=5 per group).

FIGS. 5A-5D show comparisons of time points: comparison of individual time points (FIGS. 5A and 5C) and AUCGlucose (FIGS. 5B and 5D) at study week 16. The values in FIG. 5A and FIG. 5C are given as the mean+SEM (*p<0.05, **p<0.01, ***p<0.001-HFD compared to LFD group; box p<0.001, p<0.01-HFD+vitD compared to LFD group; #p<0.05, ##p<0.01, ###p<0.001-HFD+Eloc compared to HFD group; two-way ANOVA for RM, followed by Tukey's multiple comparisons post-test; n=9-10 per group). The values in FIG. 5B and FIG. 5D are given as the mean±SEM (*p<0.05, **p<0.01, ***p<0.001, one-way ANOVA followed by Tukey's multiple comparisons post-test; n=9-10 per group).

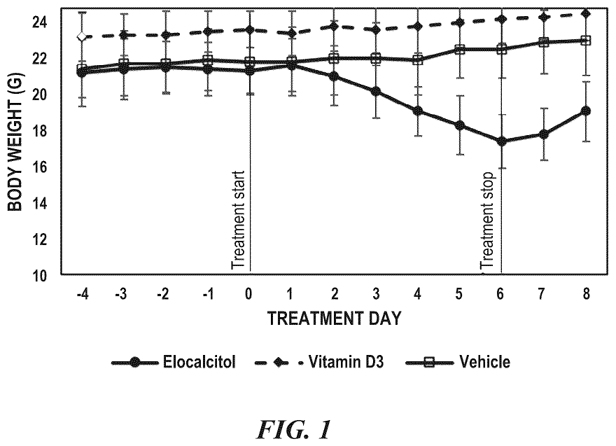

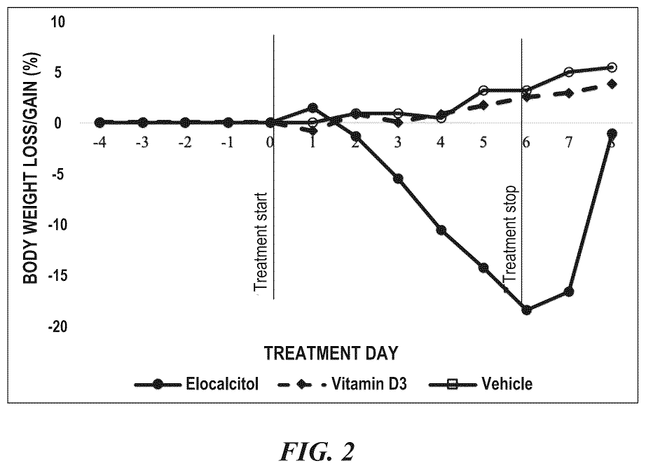

### DETAILED DESCRIPTION OF THE INVENTION

The present invention relates to methods using a vitamin D receptor agonist to treat metabolic syndrome.

### Metabolic Syndrome

Metabolic syndrome is a cluster of conditions that occur together, increasing the risk of heart disease, stroke, and type 2 diabetes. The conditions include excess body fat (visceral fat) around the waist, abnormal cholesterol or triglyceride levels (dyslipidemia), increased blood pressure (hypertension), insulin resistance or glucose intolerance, high blood sugar (hyperglycemia), obesity, atherosclerosis, diabetes mellitus, and hepatic steatosis. Metabolic syndrome is a result of chronic nutrient excess. According to the National Cholesterol Education Program (NCEP) Adult Treatment Panel III (ATP III; 2011) definition metabolic syndrome is present if at least three or more of the following criteria are met:
(i) visceral fat reflected in disproportionate fat tissue in and around the abdomen;
(ii) atherogenic dyslipidemia (high triglycerides (≥150 mg/dL), high LDL cholesterol, and low HDL cholesterol (≤50 mg/dL));
(iii) high blood pressure (≥130/85 mmHg); and insulin resistance or glucose intolerance (the inability to properly use insulin or blood sugar, respectively).

These metabolic syndrome conditions are accompanied by a chronic proinflammatory state which is reflected in higher than normal levels of high-sensitivity C-reactive protein (CRP) in the blood (Ridker et al., 2003). People with metabolic syndrome are at increased risk of type 2 diabetes, coronary heart disease, heart failure, other diseases related to plaque build ups in artery walls (e.g., stroke, myocardial infarction and peripheral vascular disease) and non-alcoholic steatohepatitis (NASH) which is the main reason for liver cirrhosis and hepatocellular carcinoma.

### Vitamin D Receptor Agonists

Elocalcitol is a non-hypercalcemic vitamin D analogue and a high-affinity vitamin D receptor agonist that increases bone metabolism (Peleg et al., 2002), has anti-proliferative and anti-inflammatory effects. It inhibits NF-κB by blocking the nuclear translocation of the p65 subunit (Penna et al., 2009). As a result, elocalcitol has been studied in humans as an experimental drug for a number of indications including overactive bladder, male infertility, chronic non-bacterial prostatitis, benign prostate hyperplasia, and adult osteoporosis. Elocalcitol is among the least hypercalcemic Vitamin D receptor agonists (Nagpal et al., 2005), it is generally accepted that elocalcitol is safe for adult humans for long treatment periods up to an oral daily dose of 300 μg (Montorsi et al., 2008).

Disclosed herein are methods using a vitamin D receptor agonist to treat metabolic syndrome. In some embodiments, one or more symptoms of metabolic syndrome may be ameliorated by the administration of a vitamin D receptor agonist or a pharmaceutical composition comprising a vitamin D receptor agonist. Also provided is the use of a vitamin D receptor agonist for the manufacture of a medicament to treat metabolic syndrome. In some embodiments, the vitamin D receptor agonist of the invention is elocalcitol.

In some embodiments, the vitamin D receptor agonist useful for the present invention is a vitamin D analogue, elocalcitol, or a pharmaceutically-acceptable salt thereof. Elocalcitol is a synthetic, biologically active vitamin D analogue with modifications to the side chain and A ring.

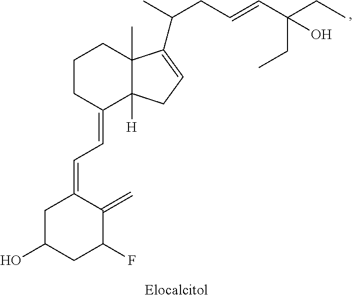 [SMILES: C=C1/C(=C\C=C2/CCCC3(C)C(C(C)C/C=C/C(O)(CC)CC)=CCC23)CC(O)CC1F]

Examples of some vitamin D receptor agonists suitable for the present invention include those of Formula 1-01 to 1-57; in which I-57 is preferred.

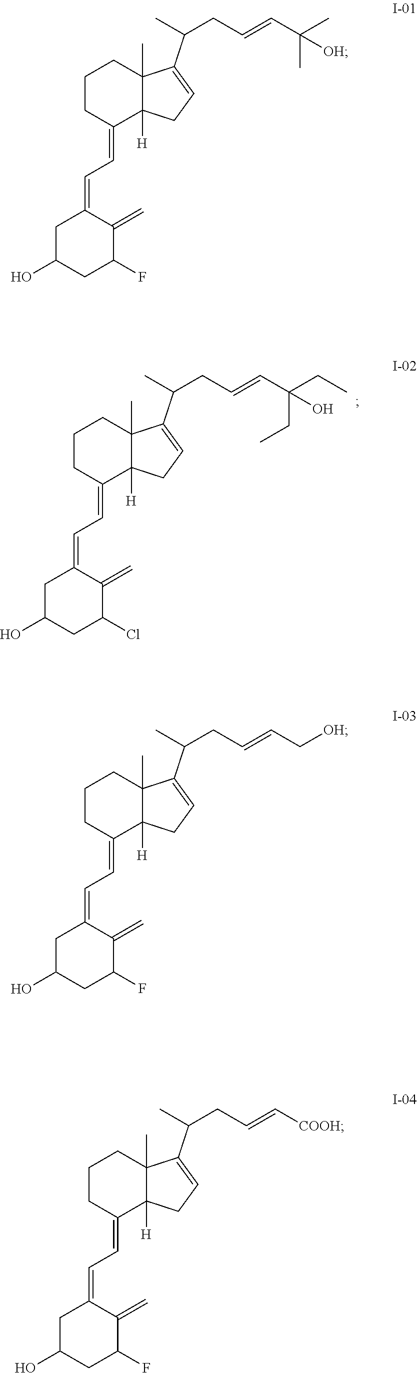 [SMILES: C=C1/C(=C\C=C2/CCCC3(C)C(C(C)C/C=C/C(C)(C)O)=CCC23)CC(O)CC1F ; C=C1/C(=C\C=C2/CCCC3(C)C(C(C)C/C=C/C(O)(CC)CC)=CCC23)CC(O)CC1Cl ; C=C1/C(=C\C=C2/CCCC3(C)C(C(C)C/C=C/CO)=CCC23)CC(O)CC1F ; C=C1/C(=C\C=C2/CCCC3(C)C(C(C)C/C=C/C(=O)O)=CCC23)CC(O)CC1F]

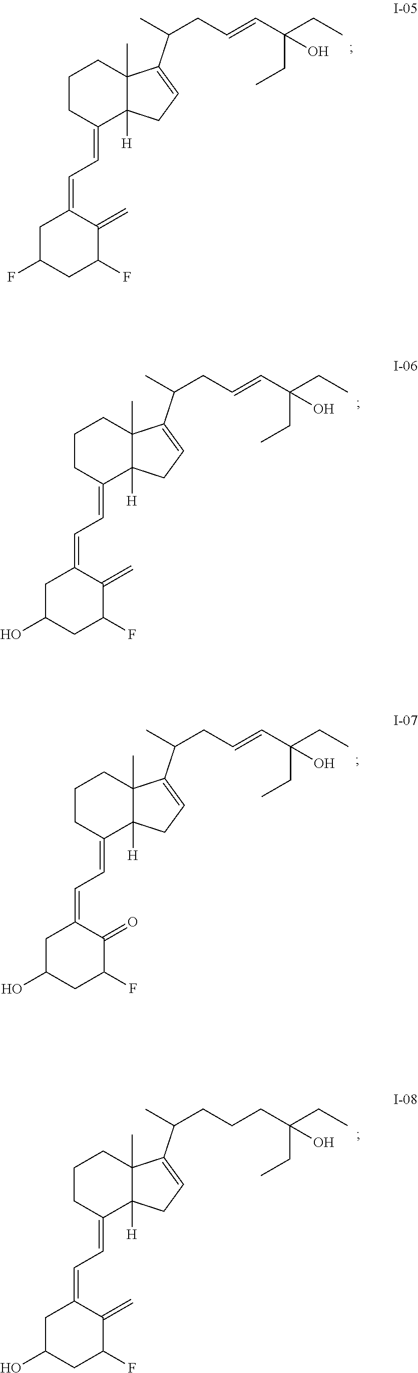 [SMILES: C=C1/C(=C\C=C2/CCCC3(C)C(C(C)C/C=C/C(O)(CC)CC)=CCC23)CC(F)CC1F ; C=C1/C(=C\C=C2/CCCC3(C)C(C(C)C/C=C/C(O)(CC)CC)=CCC23)CC(O)CC1F ; CCC(O)(/C=C/CC(C)C1=CCC2/C(=C/C=C3/CC(O)CC(F)C3=O)CCCC12C)CC ; C=C1/C(=C\C=C2/CCCC3(C)C(C(C)CCCC(O)(CC)CC)=CCC23)CC(O)CC1F]

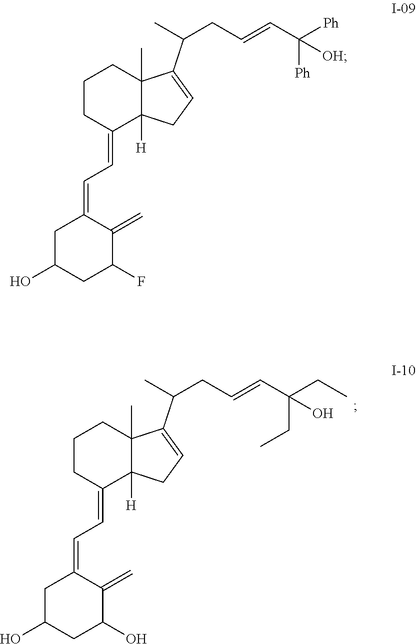 [SMILES: C=C1/C(=C\C=C2/CCCC3(C)C(C(C)C/C=C/C(O)(c4ccccc4)c4ccccc4)=CCC23)CC(O)CC1F ; C=C1/C(=C\C=C2/CCCC3(C)C(C(C)C/C=C/C(O)(CC)CC)=CCC23)CC(O)CC1O]

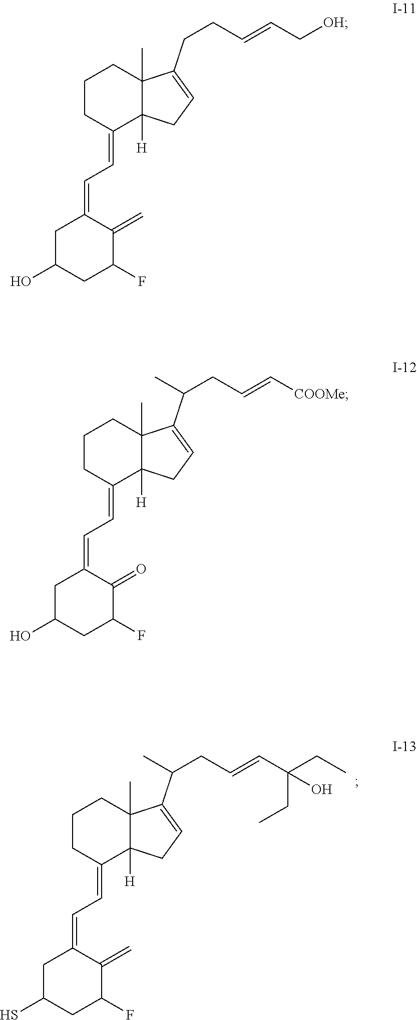 [SMILES: C=C1/C(=C\C=C2/CCCC3(C)C(CC/C=C/CO)=CCC23)CC(O)CC1F ; COC(=O)/C=C/CC(C)C1=CCC2/C(=C/C=C3/CC(O)CC(F)C3=O)CCCC12C ; C=C1/C(=C\C=C2/CCCC3(C)C(C(C)C/C=C/C(O)(CC)CC)=CCC23)CC(S)CC1F]

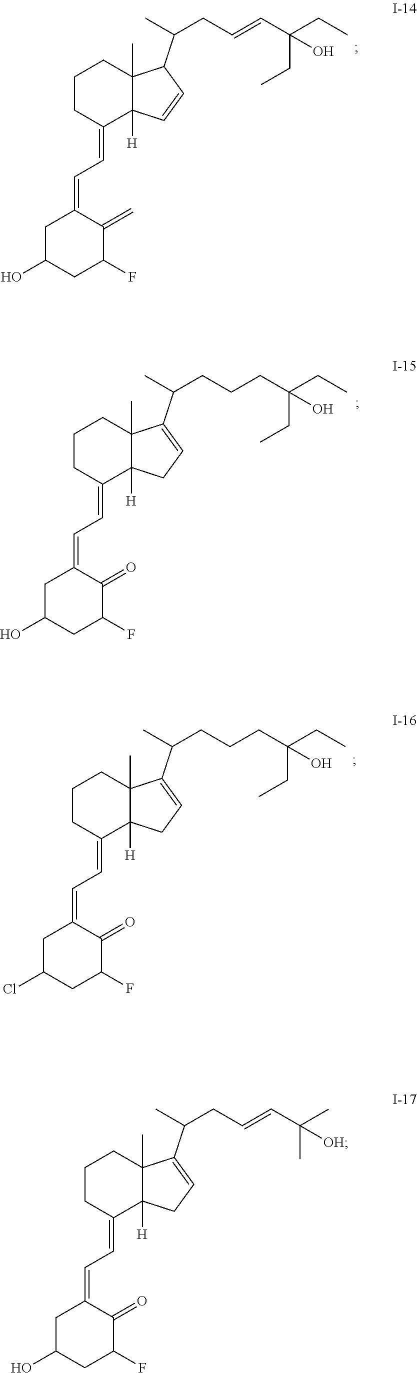 [SMILES: C=C1/C(=C\C=C2/CCCC3(C)C2C=CC3C(C)C/C=C/C(O)(CC)CC)CC(O)CC1F ; CCC(O)(CC)CCCC(C)C1=CCC2/C(=C/C=C3/CC(O)CC(F)C3=O)CCCC12C ; CCC(O)(CC)CCCC(C)C1=CCC2/C(=C/C=C3/CC(Cl)CC(F)C3=O)CCCC12C ; CC(C/C=C/C(C)(C)O)C1=CCC2/C(=C/C=C3/CC(O)CC(F)C3=O)CCCC12C]

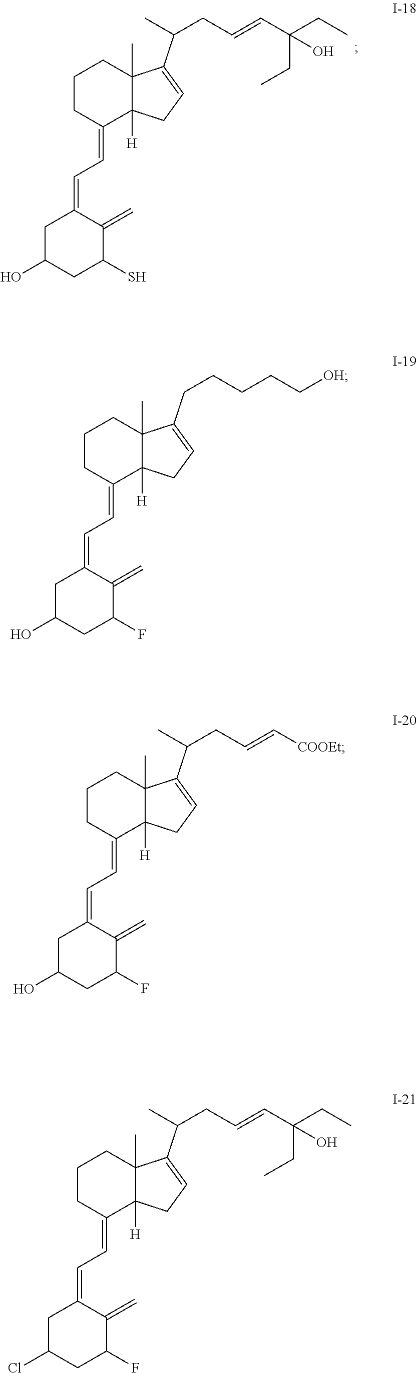 [SMILES: C=C1/C(=C\C=C2/CCCC3(C)C(C(C)C/C=C/C(O)(CC)CC)=CCC23)CC(O)CC1S ; C=C1/C(=C\C=C2/CCCC3(C)C(CCCCCO)=CCC23)CC(O)CC1F ; C=C1/C(=C\C=C2/CCCC3(C)C(C(C)C/C=C/C(=O)OCC)=CCC23)CC(O)CC1F ; C=C1/C(=C\C=C2/CCCC3(C)C(C(C)C/C=C/C(O)(CC)CC)=CCC23)CC(Cl)CC1F]

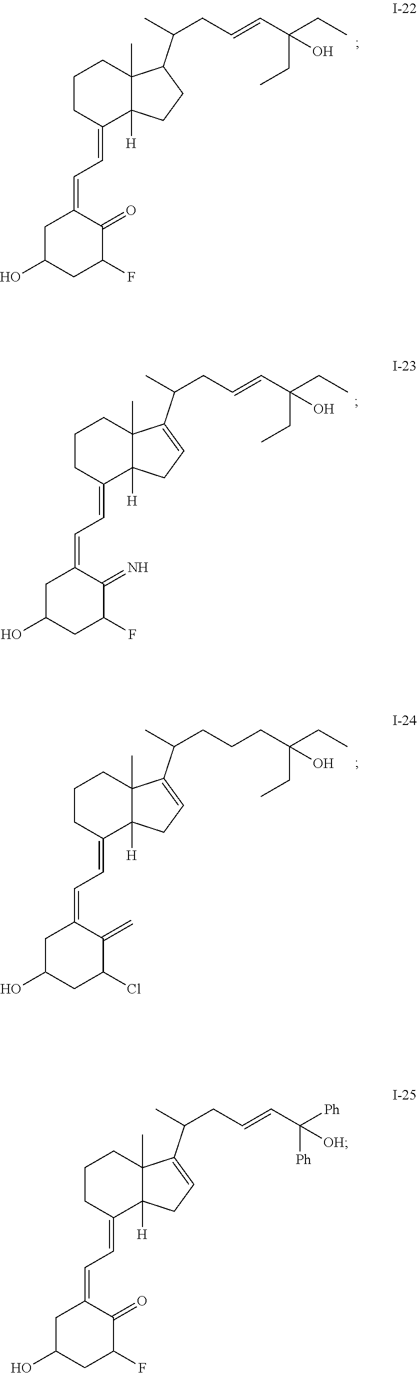 [SMILES: CCC(O)(/C=C/CC(C)C1CCC2/C(=C/C=C3/CC(O)CC(F)C3=O)CCCC21C)CC ; CCC(O)(/C=C/CC(C)C1=CCC2/C(=C/C=C3/CC(O)CC(F)C3=N)CCCC12C)CC ; C=C1/C(=C\C=C2/CCCC3(C)C(C(C)CCCC(O)(CC)CC)=CCC23)CC(O)CC1Cl ; CC(C/C=C/C(O)(c1ccccc1)c1ccccc1)C1=CCC2/C(=C/C=C3/CC(O)CC(F)C3=O)CCCC12C]

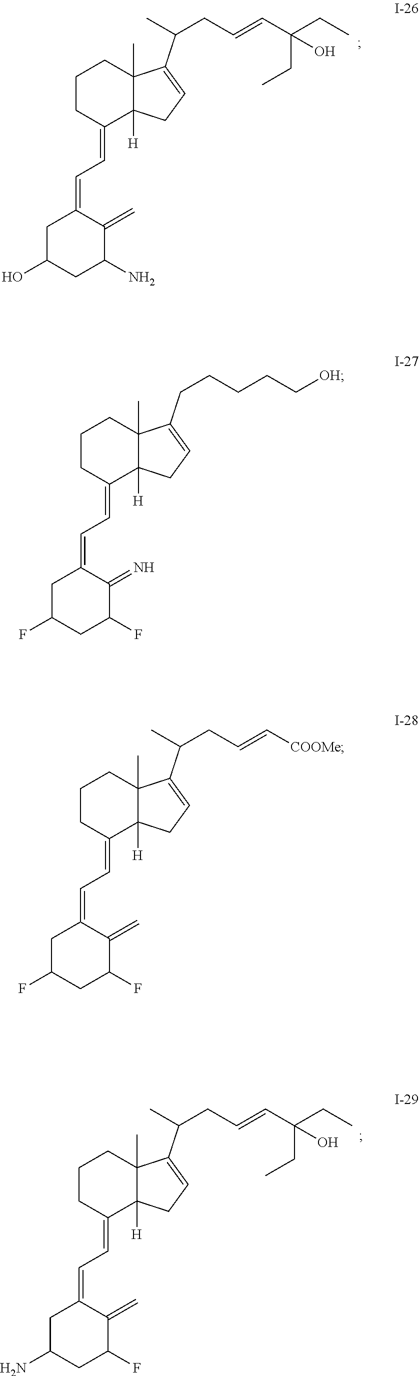 [SMILES: C=C1/C(=C\C=C2/CCCC3(C)C(C(C)C/C=C/C(O)(CC)CC)=CCC23)CC(O)CC1N ; CC12CCC/C(=C\C=C3\CC(F)CC(F)C3=N)C1CC=C2CCCCCO ; C=C1/C(=C\C=C2/CCCC3(C)C(C(C)C/C=C/C(=O)OC)=CCC23)CC(F)CC1F ; C=C1/C(=C\C=C2/CCCC3(C)C(C(C)C/C=C/C(O)(CC)CC)=CCC23)CC(N)CC1F]

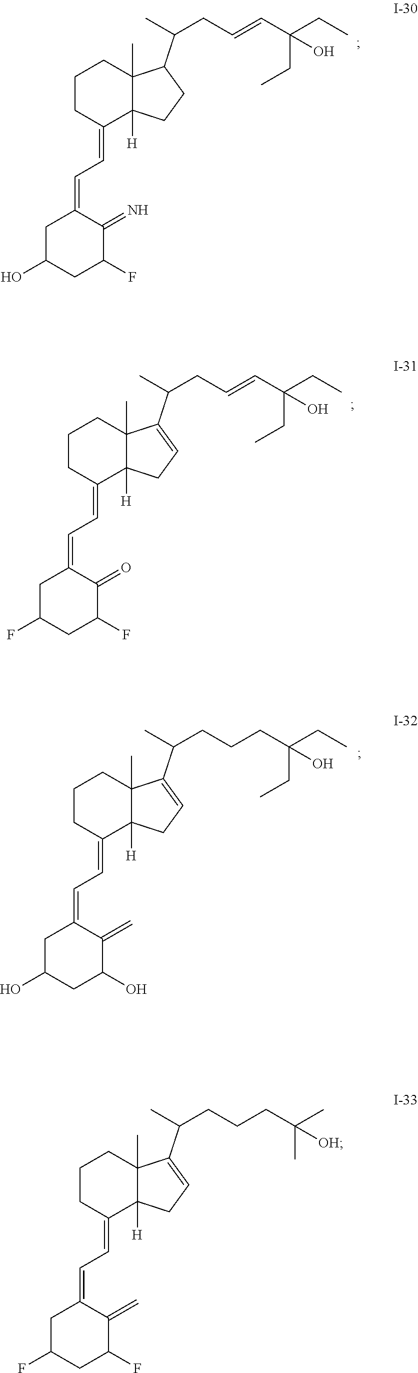 [SMILES: CCC(O)(/C=C/CC(C)C1CCC2/C(=C/C=C3/CC(O)CC(F)C3=N)CCCC21C)CC ; CCC(O)(/C=C/CC(C)C1=CCC2/C(=C/C=C3/CC(F)CC(F)C3=O)CCCC12C)CC ; C=C1/C(=C\C=C2/CCCC3(C)C(C(C)CCCC(O)(CC)CC)=CCC23)CC(O)CC1O ; C=C1/C(=C\C=C2/CCCC3(C)C(C(C)CCCC(C)(C)O)=CCC23)CC(F)CC1F]

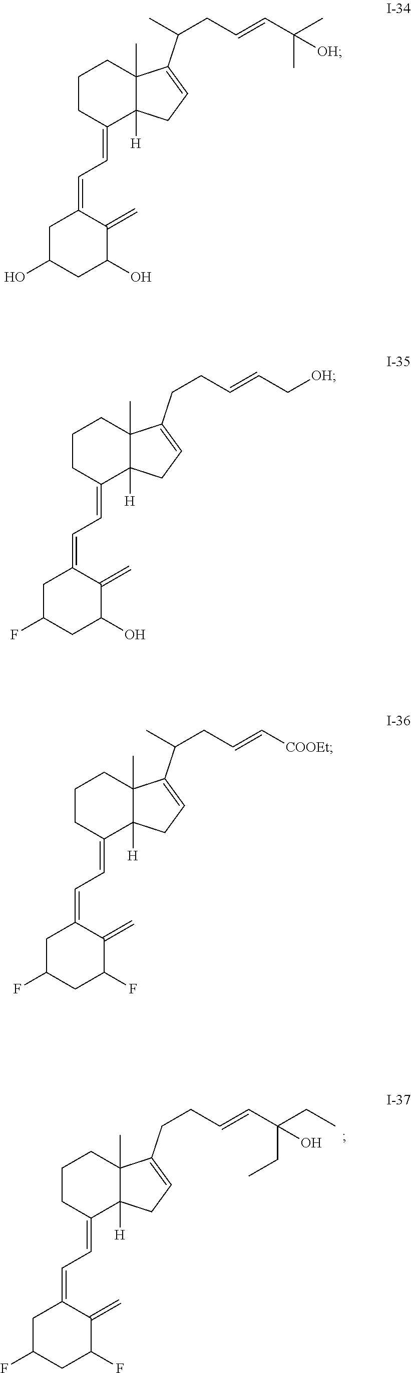 [SMILES: C=C1/C(=C\C=C2/CCCC3(C)C(C(C)C/C=C/C(C)(C)O)=CCC23)CC(O)CC1O ; C=C1/C(=C\C=C2/CCCC3(C)C(CC/C=C/CO)=CCC23)CC(F)CC1O ; C=C1/C(=C\C=C2/CCCC3(C)C(C(C)C/C=C/C(=O)OCC)=CCC23)CC(F)CC1F ; C=C1/C(=C\C=C2/CCCC3(C)C(CC/C=C/C(O)(CC)CC)=CCC23)CC(F)CC1F]

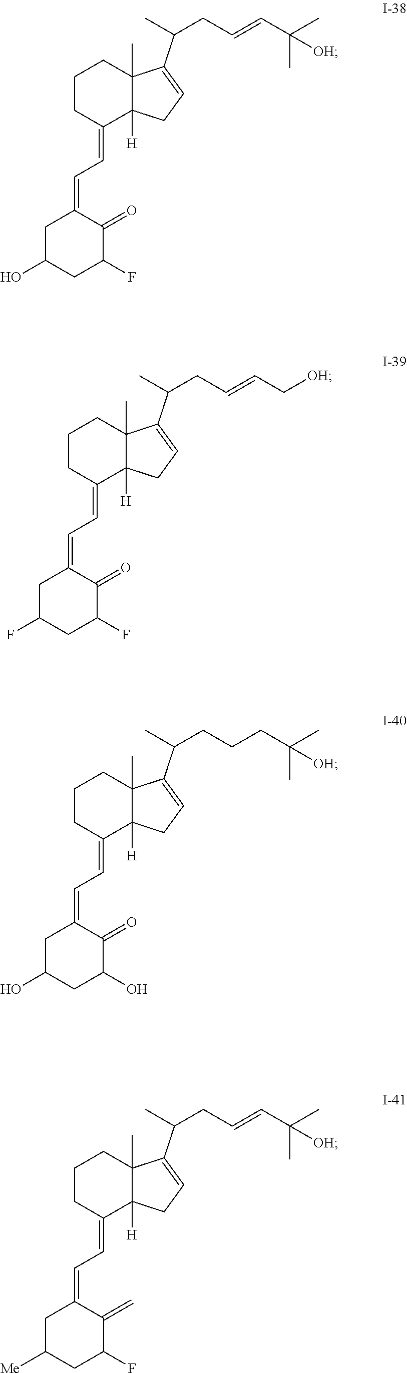 [SMILES: CC(C/C=C/C(C)(C)O)C1=CCC2/C(=C/C=C3/CC(O)CC(F)C3=O)CCCC12C ; CC(C/C=C/CO)C1=CCC2/C(=C/C=C3/CC(F)CC(F)C3=O)CCCC12C ; CC(CCCC(C)(C)O)C1=CCC2/C(=C/C=C3/CC(O)CC(O)C3=O)CCCC12C ; C=C1/C(=C\C=C2/CCCC3(C)C(C(C)C/C=C/C(C)(C)O)=CCC23)CC(C)CC1F]

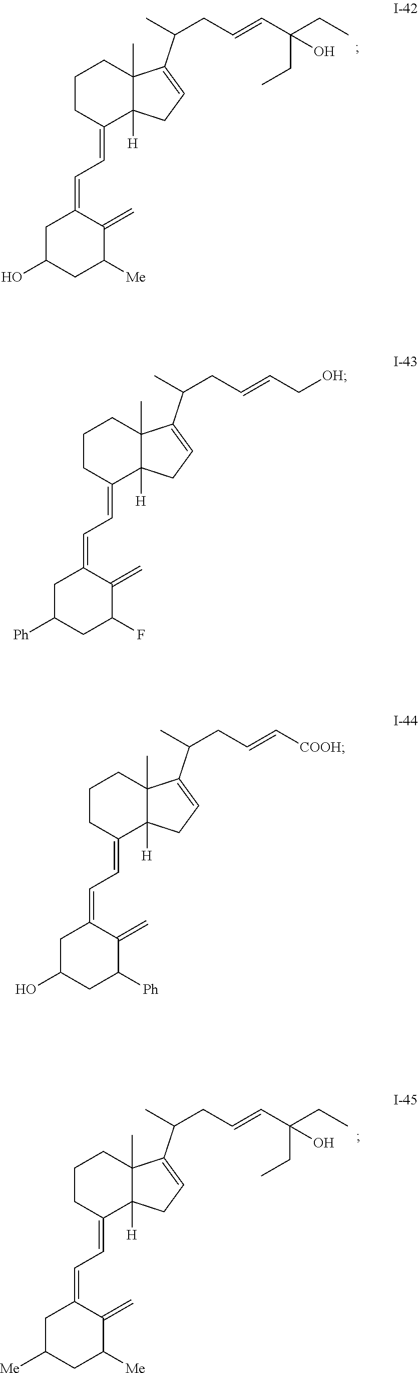 [SMILES: C=C1/C(=C\C=C2/CCCC3(C)C(C(C)C/C=C/C(O)(CC)CC)=CCC23)CC(O)CC1C ; C=C1/C(=C\C=C2/CCCC3(C)C(C(C)C/C=C/CO)=CCC23)CC(c2ccccc2)CC1F ; C=C1/C(=C\C=C2/CCCC3(C)C(C(C)C/C=C/C(=O)O)=CCC23)CC(O)CC1c1ccccc1 ; C=C1/C(=C\C=C2/CCCC3(C)C(C(C)C/C=C/C(O)(CC)CC)=CCC23)CC(C)CC1C]

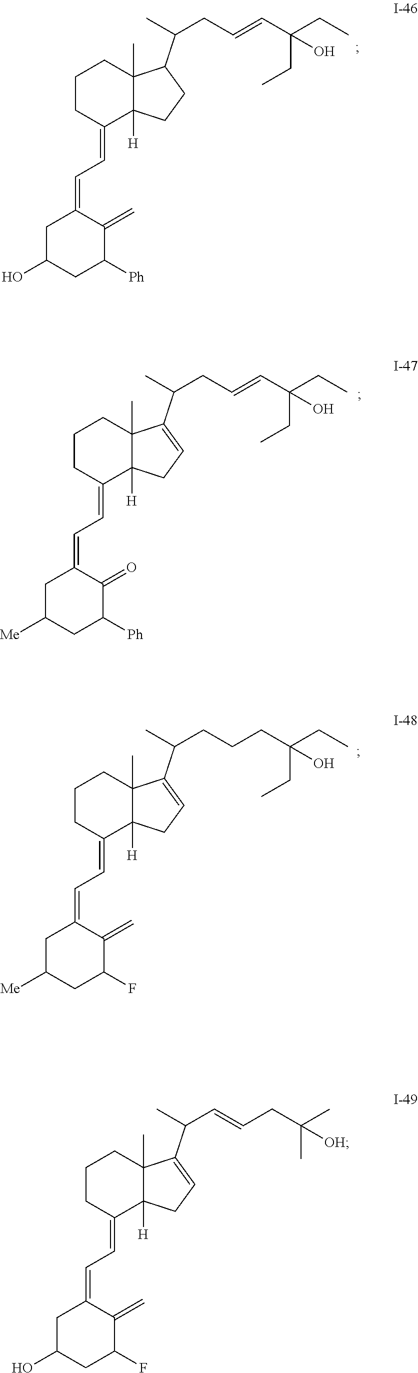 [SMILES: C=C1/C(=C\C=C2/CCCC3(C)C2CCC3C(C)C/C=C/C(O)(CC)CC)CC(O)CC1c1ccccc1 ; CCC(O)(/C=C/CC(C)C1=CCC2/C(=C/C=C3/CC(C)CC(c4ccccc4)C3=O)CCCC12C)CC ; C=C1/C(=C\C=C2/CCCC3(C)C(C(C)CCCC(O)(CC)CC)=CCC23)CC(C)CC1F ; C=C1/C(=C\C=C2/CCCC3(C)C(C(C)/C=C/CC(C)(C)O)=CCC23)CC(O)CC1F]

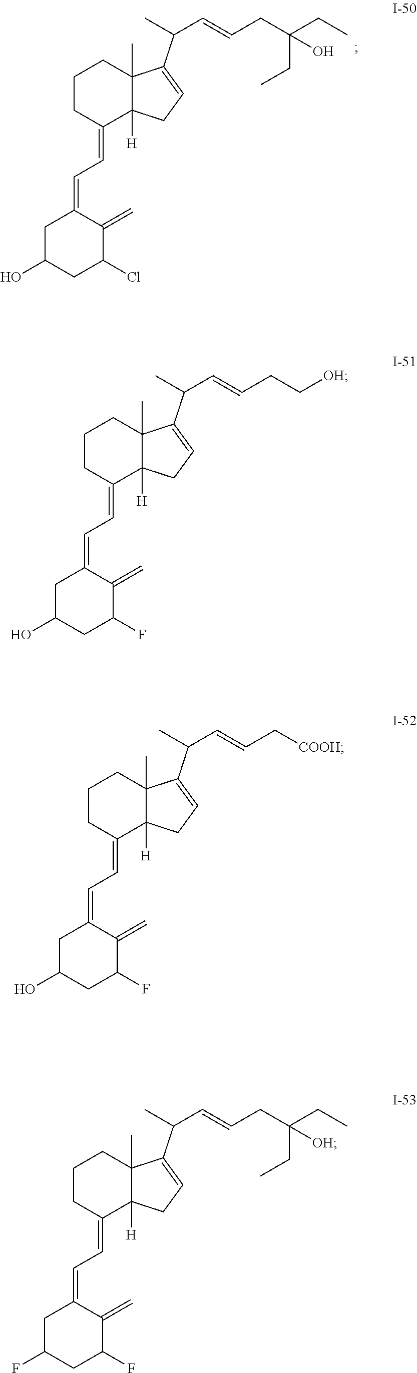 [SMILES: C=C1/C(=C\C=C2/CCCC3(C)C(C(C)/C=C/CC(O)(CC)CC)=CCC23)CC(O)CC1Cl ; C=C1/C(=C\C=C2/CCCC3(C)C(C(C)/C=C/CCO)=CCC23)CC(O)CC1F ; C=C1/C(=C\C=C2/CCCC3(C)C(C(C)/C=C/CC(=O)O)=CCC23)CC(O)CC1F ; C=C1/C(=C\C=C2/CCCC3(C)C(C(C)/C=C/CC(O)(CC)CC)=CCC23)CC(F)CC1F]

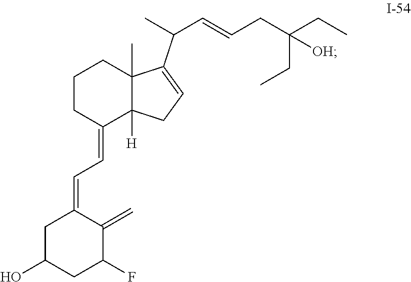 [SMILES: C=C1/C(=C\C=C2/CCCC3(C)C(C(C)/C=C/CC(O)(CC)CC)=CCC23)CC(O)CC1F]

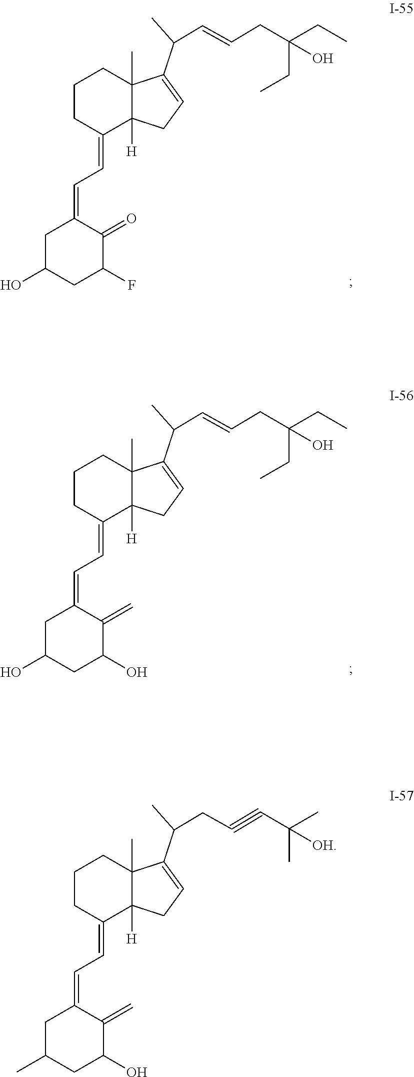

Another preferred Vitamin D receptor agonist suitable for the present invention include inecalcitol, which is the international non-proprietary name for 19-nor-9,10-seco-14βH-cholesta-5(Z),7(E)-dien-23-ino-1α,3β,25-triol-23-yne, (Formula II), or a pharmaceutically-acceptable salt thereof:

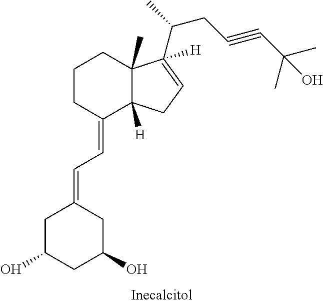

Metal salts can arise from the addition of an inorganic base to a compound described herein. Examples of suitable metals include lithium, sodium, potassium, cesium, cerium, magnesium, manganese, iron, calcium, strontium, cobalt, titanium, aluminum, copper, cadmium, and zinc. Examples of suitable metal salts include a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, and a zinc salt.

Examples of suitable ammonium salts include a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzyl amine salt, a piperazine salt, a pyridine salt, a pyrrazole salt, a pipyrrazole salt, an imidazole salt, a pyrazine salt, a pipyrazine salt, an ethylene diamine salt, an N,N′-dibenzylethylene diamine salt, a procaine salt, a chloroprocaine salt, a choline salt, a dicyclohexyl amine salt, and a N-methylglucamine salt.

### Methods of Treating Metabolic Syndrome

Disclosed herein are methods of treating metabolic syndrome using a vitamin D receptor agonist such as elocalcitol, inecalcitol, and Formula I-01 to I-57. The methods comprise administering an effective amount of a vitamin D receptor agonist, or a pharmaceutically-acceptable salt thereof, to a subject in need thereof. “An effective amount,” as used herein, is an amount effective to treat metabolic syndrome by ameliorating the pathological condition or reducing the symptoms of metabolic syndrome. In some embodiments, the effect of treatment may be determined by reducing excess body fat around the waist, reducing high cholesterol or triglyceride levels, decreasing high blood pressure, decreasing insulin resistance or glucose intolerance, decreasing high blood sugar, and reducing body weight in an obese person.

Also disclosed herein are methods for providing a prophylactic treatment of metabolic syndrome in a patient in need thereof, comprising administering to the patient an effective amount of a vitamin D analog. In some embodiments, the effect of prophylactic treatment may be determined by fasting blood glucose levels, AUC insulin levels, fasting insulin levels, cholesterol levels, AUC glucose levels, triglyceride levels, and/or body weight.

Also disclosed herein are methods for treating pre-existing abnormal levels of fasting blood glucose, AUC insulin, fasting insulin, cholesterol, AUC glucose, and/or body weight in a patient in need thereof, comprising administering to the patient an effective amount of a vitamin D receptor agonist. In some embodiments, the abnormal levels may be determined by the levels that are higher than those detected in healthy individuals. In certain other embodiments, the abnormal levels of fasting blood glucose, AUC insulin, fasting insulin, cholesterol, AUC glucose, and/or body weight are associated with metabolic syndrome. Methods for measuring the levels or extent of fasting blood glucose, AUC insulin, fasting insulin, cholesterol, AUC glucose, and/or body weight are well known in the art.

In some embodiments, the level or extent of fasting blood glucose, AUC insulin, fasting insulin, cholesterol, AUC glucose, and/or body weight is reduced by between about 5% and about 10%, between about 10% and about 20%, between about 20% and about 30%, between about 30% and about 40%, between about 40% and about 50%, between about 50% and about 60%, between about 60% and about 70%, between about 70% and about 80%, between about 80% and about 90%, or between about 90% and about 100%.

In some embodiments, the metabolic disorder may be associated with type 2 diabetes mellitus. In certain embodiments, the metabolic syndrome may be modulated by reducing visceral fat, fasting glucose, HbAlc, non-fasting glucose, improving insulin resistance, and/or reduced weight gain/weight loss. The modulation of certain glucose parameters (i.e., fasting glucose levels) may be determined using any suitable methods such as an oral glucose tolerance test.

In some embodiments, the methods of the invention can be administered, guided, and modified based on a personalized medicine approach. In some embodiments, personalized medicine provides health care methods adapted to the needs of a specific subject as opposed to methods established over medical cohorts and epidemiological studies, subsequently applied to an individual. In some embodiments, personalized medicine allows a health care provider to optimize therapy for a specific subject based on a number of factors, for example, genetics, metabolism, family history, personal history, environment, behavior, diet, lifestyle, social tendencies, and personal goals. In some embodiments, at any time before or during the therapy, a health care provider can investigate any relevant factor and use the resulting information to design or improve a therapeutic regimen. Investigation can include an assay described herein or personal counseling between the health care provider and the subject. In some embodiments, personalized medicine allows for therapy to be combined with companion diagnostic tests (such as the genotyping of the genes involved in energy metabolism (e.g., leptin, leptin receptor, genes involved in immunometabolism or any other genes leading to the clinical phenotype of metabolic syndrome) to select a population of patients who would benefit from the treatments described herein.

A subject for any of the therapeutic methods disclosed herein can be a subject in need of therapy for one or more conditions disclosed herein. The subject can be a human. Other non-limiting examples of a subject include non-human mammalian animals, such as companion animals, pets, livestock, service animals, guardian animals, laboring animals, and zoo animals.

In some embodiments, the metabolic syndrome comprises at least one condition selected from the group of excess body fat (visceral fat) around the waist, abnormal cholesterol or triglyceride levels (dyslipidemia), increased blood pressure (hypertension), insulin resistance or glucose intolerance, high blood sugar (hyperglycemia), obesity, atherosclerosis, diabetes mellitus, and hepatic steatosis. In some embodiments, the condition is excess body fat (visceral fat) around the waist. In some embodiments, the condition is abnormal cholesterol or triglyceride levels (dyslipidemia). In some embodiments, the condition is increased blood pressure (hypertension). In some embodiments, the condition is high blood sugar (hyperglycemia). In some embodiments, the condition is diabetes mellitus. In some embodiments, the condition is obesity.

In some embodiments, the vitamin D receptor agonist is administered subcutaneously, orally, topically, transdermally, intradermally, parenterally, intravenously, intraarterially, intramuscularly, intracolonicly, intraventricularly, intraspinally, intraperitoneally, intranasally, intramuscularly, sublingually, buccally, mucosally, by aerosol, or by suppository. In preferred embodiments, the vitamin D receptor agonist is administered subcutaneously or orally.

In some embodiments, the dose of the vitamin D receptor agonist is administered between about 0.1 mg/day and about 10 mg/day. In some embodiments, the dose is between about 0.1 mg/day and about 1 mg/day by oral administration. In some embodiments, the dose is between about 0.1 mg/week and about 10 mg/week. In some embodiments, the dose is a weekly extended-release dose, for example, by extended-release subcutaneous injection. In some embodiments, the dose is between about 0.1 mg/month to about 300 mg/month. In some embodiments, the dose is a monthly extended-release dose, for example, by extended-release subcutaneous injection. In some embodiments, the dose is between about 0.1 mg every 6 months to about 2 g every 6 months.

In some embodiments, a vitamin D receptor agonist such as elocalcitol may be administered at therapeutic doses over prolonged periods without affecting calcium levels. In some embodiments, elocalcitol is administered to adult humans for long treatment periods up to a daily oral dose of 150 μg, which is shown to be safe (Montorsi et al., 2008). At oral doses higher than 300 μg per day, hypercalcemia, a common side-effect of prolonged use of a vitamin D analogue, may ensue.

In some embodiments, the method further comprises administering to the subject at least one additional therapeutic agent. In some embodiments, the at least one therapeutic agent is selected from the group consisting of: glucagon-like peptide-1 (GLP-1) receptor agonists, glucose-dependent insulinotropic polypeptide (GIP) agonists, glucagon agonists, amylin agonists, farnesoid X receptor (FXR) agonists, liver X receptor (LXR) agonists, melanocortin 4 receptor (MC4R) agonists, peroxisome proliferator-activated receptor (PPAR) agonists, thyroid hormone receptor-beta (TRβ) agonists, fibroblast growth factor 21 (FGF21) analogs, activin type II receptor (ActRII) blockers, statins, sodium glucose co-transporter 2 (SGLT2) inhibitors and dipeptidyl peptidase 4 (DPP4) inhibitors.

In some embodiments, the at least one therapeutic agent is selected from the group consisting of semaglutide, liraglutide, tirzepatide, cagrilintide, lixisenatide, exenatide, albiglutide, dulaglutide, rosiglitazone, empagliflozin, dapagliflozin, canagliflozin, orlistat, bimagrumab, resmcritrom, pegozafermin, setmelanotide and metformin. In embodiments, the therapeutic agent is semaglutide.

In the combination treatment of metabolic syndrome with the vitamin D receptor analog of the present invention and another therapeutic agent, the standard dosage of another therapeutic agent is applied. For example, semaglutide is administered by 1 to 2.4 mg weekly injection, and tirzepatide is administered by 2.5-15 mg weekly injection.

In some embodiments, the subject has been diagnosed with metabolic syndrome. In embodiments, the subject has three or more conditions selected from the group consisting of: (i) visceral fat reflected in disproportionate fat tissue in and around the abdomen; (ii) atherogenic dyslipidemia (high triglycerides (≥150 mg/dL), high LDL cholesterol, and low HDL cholesterol (≤50 mg/dL)); (iii) high blood pressure (≥130/85 mmHg); and (iv) insulin resistance or glucose intolerance (the inability to properly use insulin or blood sugar, respectively).

Disclosed herein is the use of a vitamin D receptor agonist for the manufacture of a medicament for the treatment of metabolic syndrome in a subject in need thereof. Also disclosed herein is a vitamin D receptor agonist or a pharmaceutically acceptable salt thereof as a pharmaceutical compound for use in a method for preventing and/or treating metabolic syndrome in a subject. Additionally disclosed herein is a compound selected from the group consisting of compounds of formula I (elocalcitol), formula II (inecalcitol), and formula I-01 to I-57 for use in a method for treating, preventing, and/or reducing metabolic syndrome in a subject.

### Pharmaceutical Compositions

A pharmaceutical composition of a vitamin D receptor agonist can be administered to a subject along with pharmaceutical excipients or diluents. These compositions can take the form of drops, solutions, suspensions, tablets, pills, capsules, powders, sustained-, controlled-, or instant-release formulations, and other formulations known in the art. A pharmaceutical composition of the invention could be modulated using suitable excipients and diluents.

A pharmaceutical composition of the invention can be formulated in a unit dosage form, each dosage containing, for example, from about 0.01 mg to 10 g of a vitamin D receptor agonist.

In some embodiments, a unit dosage form is administered to humans, domestic pets, livestock, or other animals with a pharmaceutically acceptable diluents or excipients. In some embodiments, administration is topical, parenteral, intravenous, intraarterial, intraventricular, intraperitoneal, intranasal, intramuscular, subcutaneous, aerosol, oral, or by suppository.

In some embodiments, the dosage of pharmaceutical compositions of the present invention varies depending on the symptoms, age and body weight of the subject, the nature and severity of the disorder to be treated, the route of administration, and the form of the composition. In some embodiments, a pharmaceutical composition of the invention is administered in a single dose or in divided doses.

The combined use of multiple compounds in a pharmaceutical composition of the present invention can reduce the required dosage for any individual compound. In such combined therapy, the compounds can be delivered together or separately, simultaneously or at different times.

The pharmaceutical compositions of the present invention can be administered by various means known in the art. For oral administration, a pharmaceutical composition of the invention can be formulated as tablets, capsules, granules, powders, or syrups. Pharmaceutical compositions of the present invention can be administered parenterally as injections (intravenous, intramuscular or subcutaneous), drop infusion preparations, or suppositories.

The invention is further illustrated by the following examples, which should not be interpreted as limiting the present invention in any way.

### EXAMPLES

### Example 1: Weight Gain or Loss of Animals Upon Administration of Elocalcitol

Table 1 lists abbreviations used herein.

TABLE 1
Abbreviations.

| | |
|---|---|
| ActRII | Activin type II receptor |
| ANOVA | Analysis of variance |
| AUC | Area under the curve |
| DPP4 | Dipeptidyl peptidase 4 |
| Eloc | Elocalcitol |
| FGF21 | Fibroblast growth factor 21 |
| FXR | Farnesoid X receptor |
| g | Gram |
| GIP | Glucose-dependent insulinotropic |
| | polypeptide |
| GLP-1 | Glucagon-like peptide-1 |
| HFD | High-fat diet |
| kcal | Kilocalorie |
| kg | Kilogram |
| LFD | Low-fat diet |
| LXR | Liver X receptor |
| M | Molar |
| MC4R | Melanocortin 4 receptor |
| μg | Microgram |
| min | Minute |
| mg | Milligram |
| mL | Milliliter |
| mM | Millimolar |
| MRI | Magnetic resonance imaging |
| ng | Nanograms |
| nM | Nanomolar |
| PPAR | Peroxisome Proliferator-Activated Receptor |
| S.D. | Standard deviation |
| SEM | Standard error of the mean |
| SGLT2 | Sodium glucose co-transporter 2 |
| TRβ | Thyroid hormone receptor-beta |
| VitD | 1,25(OH)₂D₃ |

Two months old male C57BL/6J mice were housed (4-5 mice per cage) with food and water available ad libidum at a temperature control of 24-26° C. and a 12 h light/dark cycle. 3-4 days before the start of the study, the mice were randomized into 3 experimental groups (N=10 per group) using online randomization tool (Graph Pad, USA). Body weights of each animal were measured once a day during the entire study, their health conditions were assessed at the same time. Elocalcitol (100 μg/kg), vitamin D₃ (cholecalciferol,100 μg/kg), or vehicle control were administrated intraperitoneally once a day for 6 days.

By treatment Day 4, the average body weight in the elocalcitol group had dropped by 10.7%. The average decrease in body weight of the elocalcitol group reached by 18.6% by treatment Day 6. The administration of elocalcitol was stopped on Day 6 after which the average body weight of mice returned to normal within two days. No weight loss was observed in either the vitamin D₃ or vehicle control groups. No adverse changes in the health conditions of mice in any group were observed.

TABLE 2
Mean body weights (±S.D.) and relative weight gain/loss of experimental
animals in elocalcitol, vitamin D₃ and vehicle groups.

| | Elocalcitol (100 μg/kg) | | | Vitamin D₃ (100 μg/kg) | | | Vehicle | | |
|---|---|---|---|---|---|---|---|---|---|
| | | | Weight | | | Weight | | | Weight |
| Admin. | Mean | | change | Mean | | change | Mean | | change |
| day | (g) | S.D. | % | (g) | S.D. | % | (g) | S.D. | % |

| | | | | | | |
|---|---|---|---|---|---|---|
| −4 | 21.40 | 1.955 | 23.40 | 1.350 | 21.60 | 1.647 |
| N = 10 | | | | | | |
| −3 | 21.60 | 1.713 | 23.50 | 1.179 | 21.90 | 1.853 |
| N = 10 | | | | | | |
| −2 | 21.70 | 1.418 | 23.50 | 1.354 | 21.90 | 1.729 |
| N = 10 | | | | | | |
| −1 | 21.60 | 1.506 | 23.70 | 1.160 | 22.10 | 1.729 |
| N = 10 | | | | | | |

Administration start

| | | | | | | | | | |
|---|---|---|---|---|---|---|---|---|---|
| 0 | 21.50 | 1.269 | | 23.80 | 1.033 | | 22.00 | 1.886 | |
| N = 10 | | | | | | | | | |
| 1 | 21.80 | 1.476 | +1.4 | 23.60 | 1.265 | −0.84 | 22.00 | 1.944 | 0 |
| N = 10 | | | | | | | | | |
| 2 | 21.20 | 1.687 | −1.4 | 24.00 | 1.414 | +0.84 | 22.20 | 2.098 | +0.91 |
| N = 10 | | | | | | | | | |
| 3 | 20.30 | 1.494 | −5.58 | 23.80 | 1.317 | 0 | 22.20 | 1.989 | +0.91 |
| N = 10 | | | | | | | | | |
| 4 | 19.20 | 1.398 | −10.7 | 24.00 | 1.491 | +0.84 | 22.10 | 1.969 | +0.45 |
| N = 10 | | | | | | | | | |
| 5 | 18.40 | 1.647 | −14.42 | 24.20 | 1.398 | +1.68 | 22.70 | 1.636 | +3.18 |
| N = 10 | | | | | | | | | |
| 6 | 17.50 | 1.509 | −18.6 | 24.40 | 1.350 | +2.52 | 22.70 | 1.636 | +3.18 |
| N = 10 | | | | | | | | | |

Administration stopped

| | | | | | | | | | |
|---|---|---|---|---|---|---|---|---|---|
| 7 | 17.90 | 1.449 | −16.75 | 24.50 | 1.269 | +2.94 | 23.10 | 1.792 | +5.0 |
| N = 10 | | | | | | | | | |
| 8 | 19.20 | 1.687 | −1.07 | 24.70 | 1.494 | +3.78 | 23.20 | 1.989 | +5.45 |
| N = 10 | | | | | | | | | |

### Example 2. Prevention of Total Body Weight Gain

Six-week-old male C57BL/6J mice were divided into 4 experimental groups (N=15 per group) fed with either a low-fat diet (LFD) or a high-fat diet (HFD) providing 10% (3.61 kcal/kg) or 45% (4.65 kcal/kg) of total energy as fat, respectively (the main fat source was lard; ssniff Spezialdiäten GmbH): (group 1) mice fed a LFD, (group 2) mice fed a HFD, (group 3) mice fed a HFD and treated with 1,25 (OH)₂D₃ (HFD+VitD), and (4) mice fed a HFD and treated with elocalcitol (HFD+Eloc). Mice were group-housed (4-5 mice per cage) on a proper temperature control of 24-26° C. with a 12 h light/dark cycle with food and water available ad libidum. 1,25 (OH)₂D₃ (1,25-dihydroxycholecalciferol,15 μg/kg) or elocalcitol (15 μg/kg) were administrated intraperitoneally twice a week for 16 weeks to the HFD+VitD and HFD+Eloc groups, respectively, while the LFD and HFD groups were administrated the vehicle solution. Body weight was measured once a week and the amount of food and water consumed by the animals was measured once a month.

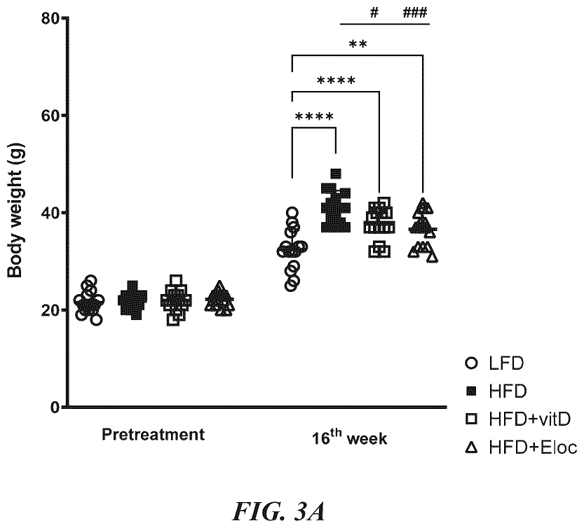

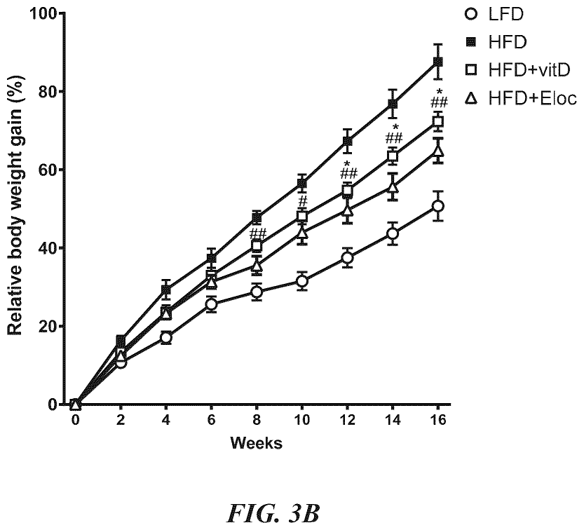

The average weight gain of mice on HFD is significantly higher than that of the LFD mice at study week 16 (FIG. 3A). Elocalcitol and 1,25 (OH)₂D₃ treatments leads to a 20% and 10% lower relative weight gain, respectively, compared to the vehicle treated HFD mice (FIGS. 3A and 3B). The inhibition of the HFD-induced weight gain by elocalcitol reaches statistical significance much earlier treated than the 1,25 (OH)₂D₃-treated mice (at study week 7 vs. study week 12, respectively (FIG. 3B).

### Example 3. Normalization of Visceral and Subcutaneous Fat Volumes

Six-week-old male C57BL/6J mice were divided into 4 experimental groups (N=15 per group) receiving either a low-fat diet (LFD) or a high-fat diet (HFD) providing either 10% (3.61 kcal/kg) or 45% (4.65 kcal/kg) of total energy as fat, respectively (the main fat source was lard; ssniff Spezialdiäten GmbH): (group 1) mice fed a LFD, (group 2) mice fed a HFD, (group 3) mice fed a HFD and treated with 1,25 (OH)₂D₃ (HFD+VitD), and (4) mice fed a HFD and treated with elocalcitol (HFD+Eloc). Mice were group-housed (4-5 mice per cage) on a proper temperature control of 24-26° C. with a 12 h light/dark cycle with food and water available ad libidum. 1,25 (OH)₂D₃ (1,25-dihydroxycholecalciferol, 15 μg/kg) or elocalcitol (15 μg/kg) were administrated intraperitoneally twice a week for 16 weeks to HFD+VitD and HFD+Eloc, respectively, while the LFD and HFD groups were administrated the vehicle solution.

Abdominal area of animals was scanned for assessing visceral and subcutaneous fat loading by using magnetic resonance imaging system (MRI, Bruker BioSpin Group, Bruker Corporations, Germany) before experiment and then once a month during the experiment. The study procedure was conducted as follows: mice (n=5 per group) were anesthetized using isoflurane (1.5-2.5% in 1.5 l/min medical oxygen) and placed on a heated animal bed throughout the MRI procedure. Scans were performed using a 9.4T Bruker BioSpec 94/21 USR system connected to a 1H circular polarized transceiver coil and running Para Vision 6.0.1® software (Bruker BioSpin Group, Bruker Corporation, Germany). Respiration was monitored using a respiration pillow (SA Instruments Inc., Stony Brook, USA) and respiration rate was maintained at between 35-70 breaths per minute. Two orientation pilot scans were performed in order to establish the position of the animal and identify anatomical landmarks relevant for planning the subsequent scan. The final Tl-weighted Bruker: RARE sequence was performed using the following parameters: repetition time (TR) 1164 ms, echo time (TE) 6 ms, flip angle 90 degrees, number of averages 2, imaging matrix 320×320×40, spatial resolution 0.125×0.125×0.5 mm. Volumes were segmented manually by an observer blinded to the experiment using ITK-SNAP (V3.8.0). A 40× zoom was used to measure visceral and subcutaneous fat volume, and renal onset was the starting point for the fat volume analysis.

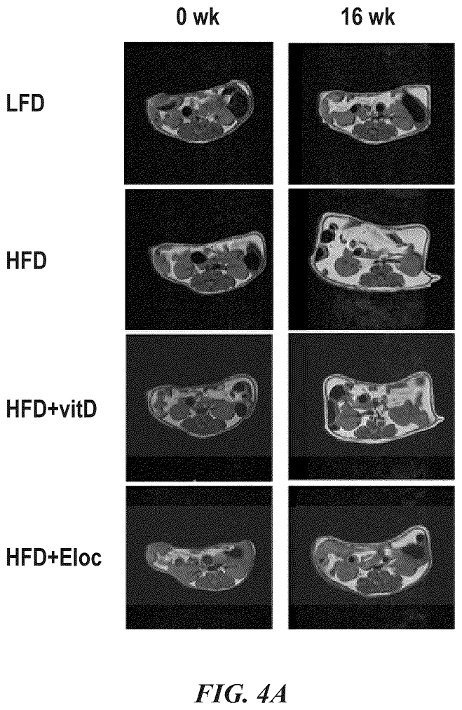

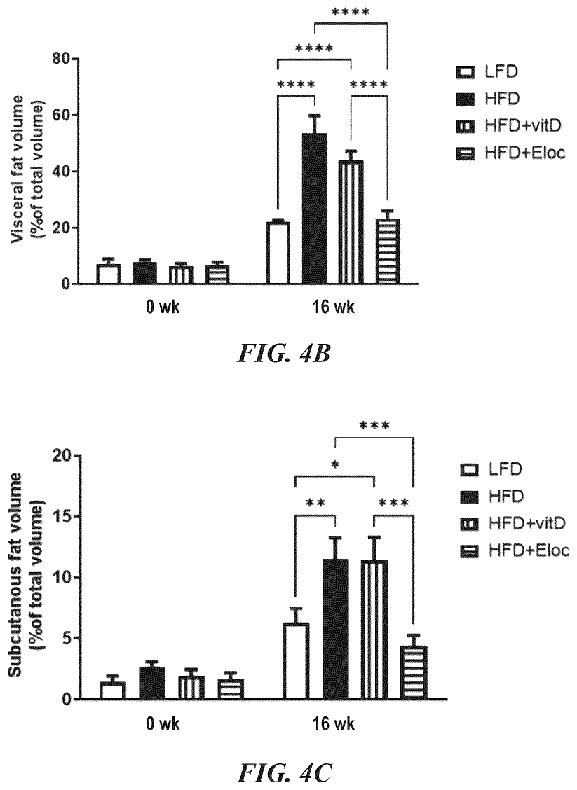

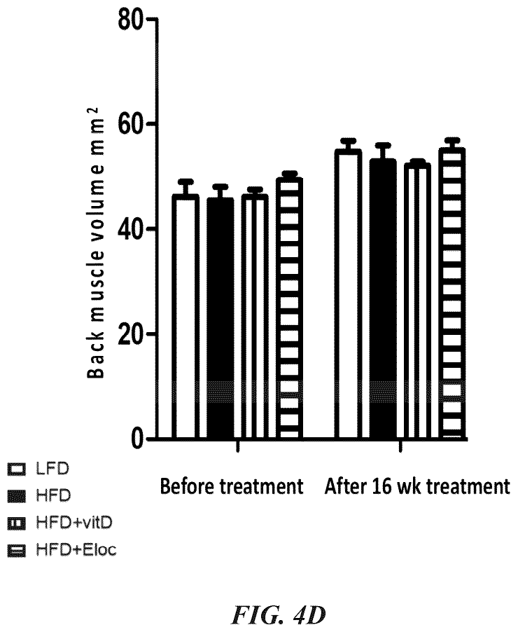

A representative image in FIG. 4 of the analysis of visceral and subcutaneous fat volumes by MRI shows that HFD leads to significantly higher visceral fat levels compared to LFD at study week 16 (****p<0.0001, two-way ANOVA, followed by Tukey's multiple comparisons post-test, n=5 per group). Treatment with elocalcitol resulted in a significant decrease in the levels of visceral and subcutaneous fat in the HFD+Eloc group compared to the HFD group (FIGS. 4; A, B and C), in fact, the elocalcitol treatment reversed the visceral and subcutaneous fat of the HFD mice to the levels observed in the LFD mice (FIGS. 4A, 4B, and 4C). 1,25 (OH)₂D₃ treatment had no statistically significant effect on neither visceral fat volume nor subcutaneous fat distribution of HFD-fed mice (FIGS. 4A and 4B). The lean mass as calculated from the volume of back muscle was preserved (FIG. 4D). The analysis of the weights of epididymal fat depositions at the study end corroborated the MRI results showing that the weights of fat deposits in the LFD and HFD+Eloc group were significantly lower than those in the HFD and HFD+vitD groups (Table 3).

TABLE 3
The effects of the diet and 1,25(OH)₂D₃ and elocalcitol on
the organ weights. The absolute organ weights (mg) and normalized
organ weights (organ weight/body weight, mg/g).

| | | | |
|---|---|---|---|
| LFD | HFD | HFD + VitD | HFD + Eloc |
| (n = 10) | (n = 10) | (n = 10) | (n = 10) |

The absolute organ weights (mg)*

| | | | | |
|---|---|---|---|---|
| Epididymal | 871.1 ± | 2019 ± | 1874 ± | 1312 ± |
| fat | 94.56 | 116.7_{****} | 88.81_{****} | 145.6_{###, ¤¤} |
| Liver | 1198 ± | 1525 ± | 1374 ± | 1412 ± |
| | 57.38 | 46.41_{*} | 97.51 | 88.64 |
| Kidneys | 322.7 ± | 345.6 ± | 323.2 ± | 362.9 ± |
| | 7.93 | 12.18 | 15.08 | 21.61 |
| Spleen | 85.60 ± | 92.80 ± | 91.20 ± | 106.2 ± |
| | 2.84 | 7.07 | 6.64 | 8.13 |

Normalized organ weights (organ weight/body weight, mg/g)

| | | | | |
|---|---|---|---|---|
| Epididymal | 27.09 ± | 50.20 ± | 49.67 ± | 36.29 ± |
| fat | 2.27 | 2.08_{****} | 2.32_{****} | 3.72_{##, ¤¤} |
| Liver | 38.76 ± | 38.22 ± | 36.03 ± | 39.45 ± |
| | 1.61 | 1.28 | 1.79 | 2.59 |
| Kidneys | 10.35 ± | 8.70 ± | 8.52 ± | 10.15 ± |
| | 0.47 | 0.42 | 0.26 | 0.65 |
| Spleen | 2.73 ± | 2.34 ± | 2.40 ± | 2.96 ± |
| | 0.08 | 0.19 | 0.13 | 0.22 |

*The data are expressed as mean ± SEM (_{*}p < 0.05, _{****}p < 0.0001- compared to LFD; _{¤¤}p < 0.01- HFD + vitD compared to HFD + Eloc; _{##}p < 0.01, _{###}p < 0.001- HFD compared to HFD + Eloc; n = 10 per group, one-way ANOVA followed by Tukey's multiple comparisons post-tests).

### Example 4. Reversal of Glucose Intolerance and Insulin Resistance

Six-week-old male C57BL/6J mice were divided into 4 experimental groups (N=15 per group) receiving either a low-fat diet (LFD) or a high-fat diet (HFD) providing either 10% (3.61 kcal/kg) or 45% (4.65 kcal/kg) of total energy as fat, respectively (the main fat source was lard; ssniff Spezialdiäten GmbH): (group 1) mice fed a LFD, (group 2) mice fed a HFD, (group 3) mice fed a HFD and treated with 1,25 (OH)₂D₃ (HFD+VitD), and (4) mice fed a HFD and treated with elocalcitol (HFD+Eloc). Mice were group-housed (4-5 mice per cage) on a proper temperature control of 24-26° C. with a 12 h light/dark cycle with food and water available ad libidum. 1,25 (OH)₂D₃ (1,25-dihydroxycholecalciferol, 15 μg/kg) or elocalcitol (15 μg/kg) were administrated intraperitoneally (i.p.) to the HFD+VitD and HFD+Eloc groups while the LFD and HFD groups were administrated the vehicle solution twice a week for 16 weeks.

To determine the effect of elocalcitol and 1,25 (OH)₂D₃ in hyperglycemia and insulin sensitivity in mice, the glucose tolerance tests (GTT) were performed at baseline and once a month during the study. For GTT, mice were fasted overnight for 10-12 h and blood glucose was measured from the tail blood using Accu-Chek Performa system glucose meter (Roche, Germany) and the baseline glucose values (0 min) were recorded. The animals were then injected intraperitoneally with glucose (2 g/kg) and after glucose injection the blood glucose was measured from tail blood according to this schedule: 15, 30, 60, 90, and 120 min.

Insulin tolerance test (ITT) was performed at the end of the study (study week 16). For ITT, mice were fasted for 6 h and blood glucose was measured from the tail blood using Accu-Chek Performa system glucose meter (Roche, Germany) and the baseline glucose values (0 min) were recorded. The animals were then injected intraperitoneally with insulin (0,5 U/kg; diluted from 100 U/ml (Novorapid, Novo Nordisk A/S) with saline 0.9%) and after glucose injection the blood glucose was measured from tail blood according to this schedule: 15, 30, 60, 90, and 120 min.

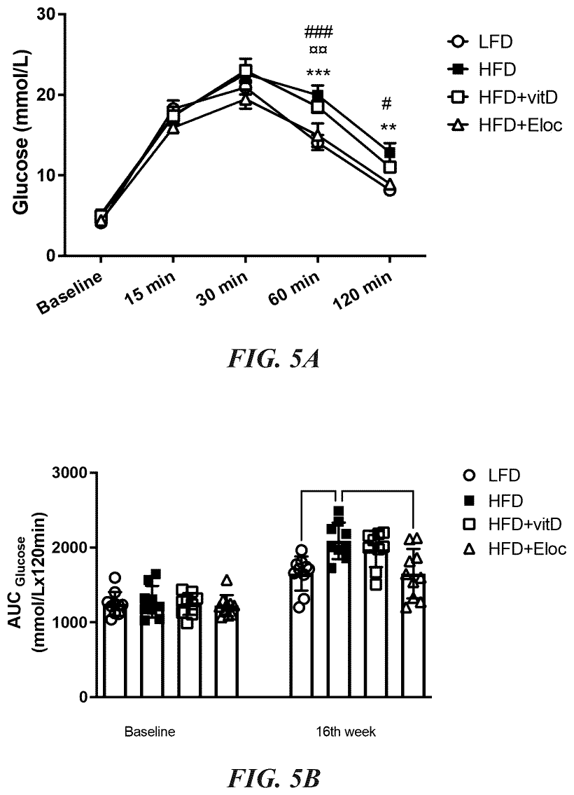

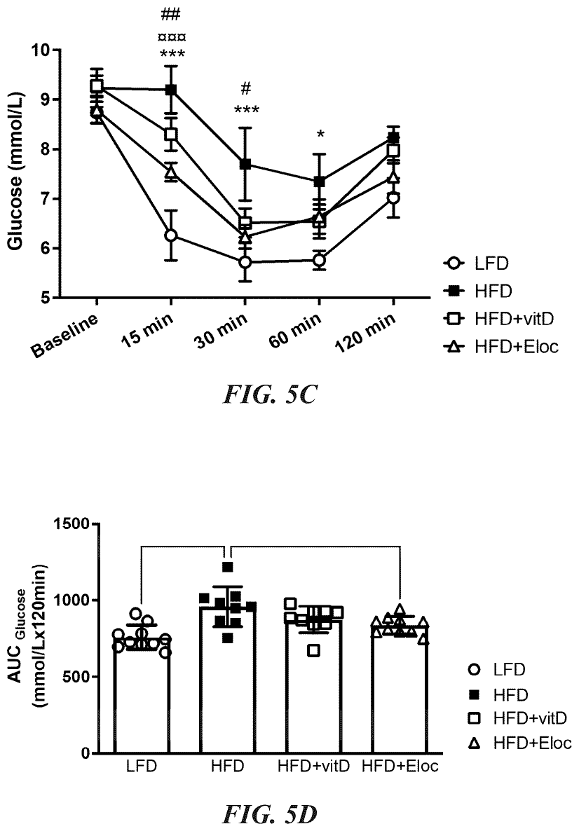

The HFD-fed mice exhibited a significantly impaired glucose tolerance compared to the LFD controls following the glucose administration (2 g/kg, i.p.) at study week 16, as determined by comparison of individual time points and the glucose area under the curve analysis (FIGS. 5A and 5B). The glucose area under the curve (glucose AUC) analysis, an index of the entire glucose excursion after the glucose load, revealed that the treatment by elocalcitol, but not 1,25 (OH)₂D₃, resulted in statistically significant improvement in the glucose tolerance of the HFD-fed mice (FIG. 5B). Insulin tolerance test results show that the treatment by elocalcitol, but not 1,25 (OH)₂D₃, reverses the increased insulin resistance observed in the HFD in a statistically significant manner (FIGS. 5C and 5D).

### Example 5. Normalization of Triglyceride and Cholesterol Levels

Six-week-old male C57BL/6J mice were divided into 4 experimental groups (N=15 per group) receiving either a low-fat diet (LFD) or a high-fat diet (HFD) providing either 10% (3.61 kcal/kg) or 45% (4.65 kcal/kg) of total energy as fat, respectively (the main fat source was lard; ssniff Spezialdiäten GmbH): (group 1) mice fed a LFD, (group 2) mice fed a HFD, (group 3) mice fed a HFD and treated with 1,25 (OH)₂D₃ (HFD+VitD), and (4) mice fed a HFD and treated with elocalcitol (HFD+Eloc). Mice were group-housed (4-5 mice per cage) on a proper temperature control of 24-26° C. with a 12 h light/dark cycle with food and water available ad libidum. 1,25 (OH)₂D₃ (1,25-dihydroxycholecalciferol, 15 μg/kg) or elocalcitol (15 μg/kg) were administrated intraperitoneally to the HFD+VitD and HFD+Eloc groups while the LFD and HFD groups were administrated the vehicle solution twice a week for 16 weeks.

The mice were deeply anesthetized with a terminal dosage of phenobarbital (200-300 mg/kg)₂₄ h after the last study treatment administration and the blood samples were collected from all groups of mice via cardiac puncture using a standardized protocol. Collected blood was allowed to coagulate before centrifugation at 2000×g for 20 min after which the serum was stored at −80° C. until the analysis.

Triglyceride levels were significantly elevated in the HFD group compared to the LFD group (p<0.05, one-way ANOVA followed by Tukey's multiple comparisons post-test, Table 3). The treatment with both elocalcitol as well as with 1,25 (OH)₂D₃ lowered the triglyceride levels similar to the ones in the LFD group, whereas the treatment with elocalcitol had a statistically significant cholesterol (the combined HDL and LDL) lowering effect (Table 4).

TABLE 4
Blood metabolic markers in mice of the LFD, HFD and HFD +
VitD and HFD + Eloc groups at study week 16.
Metabolic parameters in serum (mmol/L)

| | LFD | HFD | HFD + VitD | HFD + Eloc |
|---|---|---|---|---|
| | (n = 9) | (n = 9) | (n = 8) | (n = 9) |
| Triglycerides | 0.67 ± | 1.23 ± | 0.68 ± | 0.87 ± |
| | 0.03 | 0.25_{*} | 0.04 | 0.09 |
| Total | 2.96 ± | 4.32 ± | 4.44 ± | 3.88 ± |
| cholesterol | 0.14 | 0.27_{**} | 0.17_{**} | 0.34 |
| HDL | 2.42 ± | 3.27 ± | 3.57 ± | 2.99 ± |
| | 0.11 | 0.20_{**} | 0.09_{***} | 0.22 |
| LDL | 0.43 ± | 0.81 ± | 0.82 ± | 0.73 ± |
| | 0.04 | 0.06_{***} | 0.07_{***} | 0.08_{*} |

### Example 6. Biphasic Weight Loss Treatment Using Variable Strength Extended-Release Elocalcitol Formulations

Subjects experiencing symptoms of metabolic disease and diagnosed with obesity will be prescribed a monotherapy of a vitamin D receptor agonist, wherein the vitamin D receptor agonist is elocalcitol. Elocalcitol will initially be administered by a weekly subcutaneous injection as a prefilled syringe containing a sterile liquid extended-release formulation of elocalcitol in polar lipid phase consisting of a mix of phospholipid, diacylglycerol, ethanol and buffer yielding a release of the daily effective dose of about 0.6 to 1.2 mg of elocalcitol. The initial treatment with elocalcitol will be continuous for up to six months. Thereafter, elocalcitol will be administered by a quarterly subcutaneous injection as a prefilled syringe containing a sterile liquid extended-release formulation of elocalcitol in polar lipid phase consisting of a mix of phospholipid, diacylglycerol, ethanol and citrate buffer or in-situ polymeric gel extended delivery system yielding a release of a daily effective dose of about 0.3 mg of elocalcitol. Therapy will be continuous for as long as therapeutic efficacy is detectable. The subject will be monitored throughout the course of therapy to determine therapeutic efficacy, minimized side-effects (e.g., hypercalcemia), and any need for changes to dosing or the prescribed therapeutic regimen.

### Example 7. Biphasic Weight Loss Treatment Using Variable Strength Extended-Release Elocalcitol Formulations in Combination with GLP-1 Agonist

Subjects experiencing symptoms of metabolic disease and diagnosed with obesity will be prescribed a monotherapy of a vitamin D receptor agonist, wherein the vitamin D receptor agonist is elocalcitol. Elocalcitol will initially be administered by a weekly subcutaneous injection as a prefilled syringe containing a sterile liquid extended-release formulation of elocalcitol in polar lipid phase consisting of a mix of phospholipid, diacylglycerol, ethanol and buffer yielding a release of the daily effective dose of about 0.6 to 1.2 mg of elocalcitol. Simultaneously, the subject will be treated with weekly subcutaneous injections of 2.4 mg of semaglutide. The initial treatment with this combination of elocalcitol and semaglutide will be continuous for up to six months. Thereafter, elocalcitol will be administered by a quarterly subcutaneous injection as a prefilled syringe containing a sterile liquid extended-release formulation of elocalcitol in polar lipid phase consisting of a mix of phospholipid, diacylglycerol, ethanol and citrate buffer or in-situ polymeric gel extended delivery system yielding a release of a daily effective dose of about 0.3 mg of elocalcitol. Therapy will be continuous for as long as therapeutic efficacy is detectable. The subject will be monitored throughout the course of therapy to determine therapeutic efficacy, minimized side-effects (e.g., hypercalcemia), and any need for changes to dosing or the prescribed therapeutic regimen.

### Example 8. Biphasic Treatment of Glucose Intolerance Using Variable Strength Extended-Release Elocalcitol Formulations

Subjects experiencing symptoms of metabolic disease and diagnosed with type 2 diabetes will be prescribed a monotherapy of a vitamin D receptor agonist, wherein the vitamin D receptor agonist is elocalcitol. Elocalcitol will initially be administered by a weekly subcutaneous injection as a prefilled syringe containing a sterile liquid extended-release formulation of elocalcitol in polar lipid phase consisting of a mix of phospholipid, diacylglycerol, ethanol and buffer yielding a release of the daily effective dose of about 0.6 to 1.2 mg of elocalcitol. The initial treatment with elocalcitol will be continuous for up to six months. Thereafter, elocalcitol will be administered by a quarterly subcutaneous injection as a prefilled syringe containing a sterile liquid extended-release formulation of elocalcitol in polar lipid phase consisting of a mix of phospholipid, diacylglycerol, ethanol and citrate buffer or in-situ polymeric gel extended delivery system yielding a release of a daily effective dose of about 0.3 mg of elocalcitol. Therapy will be continuous for as long as therapeutic efficacy is detectable. The subject will be monitored throughout the course of therapy to determine therapeutic efficacy, minimized side-effects (e.g., hypercalcemia), and any need for changes to dosing or the prescribed therapeutic regimen.

### Exemplary Embodiments

In an embodiment, disclosed herein is a method of treating or reducing effects of metabolic syndrome in a subject, the method comprising: administering to the subject in need thereof an effective amount of the vitamin D receptor agonist, or a pharmaceutically acceptable salt thereof, wherein the vitamin D receptor agonist is selected from the group consisting of a compound of elocalcitol, inecalcitol, I-01, I-02, I-03, I-04, I-05, I-06, I-07, I-08, I-09, I-10, I-11, I-12, I-13, I-14, I-15, I-16, I-17, I-18, I-19, I-20, I-21, I-22, I-23, I-24, I-25, I-26, I-27, I-28, I-29, I-30, I-31, I-32, I-33, I-34, I-35, I-36, I-37, I-38, I-39, I-40, I-41, I-42, I-43, I-44, I-45, I-46, I-47, I-48, I-49, I-50, I-51, I-52, I-53, I-54, I-55, I-56 and I-57, and wherein the effective amount of vitamin D receptor agonist does not exceed a threshold over which hypercalcemia is induced.

In an embodiment, the vitamin D receptor agonist is elocalcitol, inecalcitol, or Formula I-57.

In an embodiment, the metabolic syndrome comprises at least one condition selected from the group of excess body fat (visceral fat) around the waist, abnormal cholesterol or triglyceride levels (dyslipidemia), increased blood pressure (hypertension), insulin resistance or glucose tolerance, high blood sugar (hyperglycemia), obesity, atherosclerosis, diabetes mellitus, and hepatic steatosis.

In an embodiment, the condition is excess body fat (visceral fat) around the waist.

In an embodiment, the condition is abnormal cholesterol or triglyceride levels (dyslipidemia).

In an embodiment, the condition is increased blood pressure (hypertension).

In an embodiment, the condition is high blood sugar (hyperglycemia).

In an embodiment, the condition is diabetes mellitus.

In an embodiment, the condition is obesity.

In an embodiment, the threshold is less than or equal to 1,200 μg per day.

In an embodiment, the threshold is greater than or equal to about 150 μg per day and less than or equal to about 1,200 μg per day.

In an embodiment, the vitamin D receptor agonist is administered topically, transdermally, intradermally, parenterally, intravenously, intraarterially, subcutaneously, intramuscularly, intracranially, intracolonicly, intraorbitally, ophthalmicly, intraventricularly, intracapsulary, intraspinally, intracisternally, intraperitoneally, intranasally, intramuscularly, subcutaneously, sublingually, buccally, mucosally, by aerosol, orally, or by suppository.

In an embodiment, the vitamin D receptor agonist is administered subcutaneously.

In an embodiment, the method further comprises administering to the subject one or more pharmaceutically acceptable excipients.

In an embodiment, the method further comprises administering to the subject at least one therapeutic, or a pharmaceutically acceptable salt thereof.

In an embodiment, the at least one therapeutic agent is selected from the groupconsisting of: glucagon-like peptide-1 (GLP-1) receptor agonists, glucose-dependent insulinotropic polypeptide (GIP) agonists, glucagon agonists, amylin agonists, farnesoid X receptor (FXR) agonists, liver X receptor (LXR) agonists, melanocortin 4 receptor (MC4R) agonists, peroxisome proliferator-activated receptor (PPAR) agonists, thyroid hormone receptor-beta (TRβ) agonists, fibroblast growth factor 21 (FGF21) analogs, activin type II receptor (ActRII) blockers, statins, sodium glucose co-transporter 2 (SGLT2) inhibitors and dipeptidyl peptidase 4 (DPP4) inhibitors.

In an embodiment, the at least one therapeutic agent is selected from the group consisting of semaglutide, liraglutide, tirzepatide, cagrilintide, lixisenatide, exenatide, albiglutide, dulaglutide, rosiglitazone, empagliflozin, dapagliflozin, canagliflozin, orlistat, bimagrumab, resmeritrom, pegozafermin, setmelanotide and metformin.

In an embodiment, the at least one therapeutic agent is administered to the subject at a dose of less than or equal to about 1000 milligrams (mg).

In an embodiment, the vitamin D receptor agonist is administered to the subject at a dose less than or equal to about 1000 milligrams (mg).

In an embodiment, the subject has metabolic syndrome.

### REFERENCES

Baker R. G. et al., Cell Metab. 2011; 13 (1): 11-22.
Chawla A. et al., Nat. Rev. Immunol. 2011; 11 (11): 738-749.
He Y. et al., Trends Biochem. Sci. 2016; 41 (12): 1012-1021.
Hotamisligil G. S., Immunity 2017; 47 (3): 406-420.
Hotamisligil G. S., Nature 2006; 444 (7121): 860-867.
Montorsi F. et al., J. Urol. 2008; 179 (suppl): 700; Abstract 2035.
Nagpal S. et al., Endocrine Rev. 2005; 26 (5): 662-687.
National Cholesterol Education Program (NCEP) Adult Treatment Panel III (ATP III), JAMA 2001; 285 (19): 2486-2497.
Peleg S. et al., Endocrinology 2002; 143 (5): 1625-1636.
Penna G. et al., Prostate 2009; 69 (5): 480-493.
Ridker P. M. et al., Circulation 2003; 107 (3): 391-397.
Shi H. et al., J. Clin. Invest. 2006; 116 (11): 3015-3025.
Van Saklayen M. G., Curr. Hypertens. Rep. 2018; 20 (2): 12.

1. A method of treating metabolic syndrome in a subject, the method comprising: administering to a subject in need thereof an effective amount of a vitamin D receptor agonist, or a pharmaceutically acceptable salt thereof,
wherein the vitamin D receptor agonist is selected from the group consisting of: elocalcitol, inecalcitol, a compound of Formula I-01, I-02, I-03, I-04, I-05, I-06, I-07, I-08, I-09, I-10, I-11, I-12, I-13, I-14, I-15, I-16, I-17, I-18, I-19, I-20, I-21, I-22, I-23, I-24, I-25, I-26, I-27, I-28, I-29, I-30, I-31, I-32, I-33, I-34, I-35, I-36, I-37, I-38, I-39, I-40, I-41, I-42, I-43, I-44, I-45, I-46, I-47, I-48, I-49, I-50, I-51, I-52, I-53, I-54, I-55, I-56, and I-57.

2. The method of claim 1, wherein the vitamin D receptor agonist is elocalcitol.

3. The method of claim 1, wherein the vitamin D receptor agonist is inecalcitol.

4. The method of claim 1, wherein the vitamin D receptor agonist is Formula I-57.

5. The method of claim 1, wherein the metabolic syndrome comprises at least one condition selected from the group consisting of: excess body fat around the waist, abnormal cholesterol or triglyceride levels, increased blood pressure, insulin resistance or glucose intolerance, high blood sugar, obesity, atherosclerosis, diabetes mellitus, and hepatic steatosis.

6. The method of claim 5, wherein the condition is excess body fat around the waist.

7. The method of claim 5, wherein the condition is abnormal cholesterol or triglyceride levels.

8. The method of claim 5, wherein the condition is increased blood pressure.

9. The method of claim 5, wherein the condition is high blood sugar.

10. The method of claim 5, wherein the condition is diabetes mellitus.

11. The method of claim 5, wherein the condition is obesity.

12. The method of claim 1, wherein the vitamin D receptor agonist is administered orally or subcutaneously.

13. The method of claim 1, further comprising administering at least one therapeutic agent selected from the group consisting of: semaglutide, liraglutide, tirzepatide, cagrilintide, lixisenatide, exenatide, albiglutide, dulaglutide, rosiglitazone, empagliflozin, dapagliflozin, canagliflozin, orlistat, bimagrumab, resmeritrom, pegozafermin, setmelanotide, and metformin.
